(19) United States
(12) Patent Application Publication
(10) Pub. No.: US 2020/0373019 A1
(43) Pub. Date: Nov. 26, 2020

(54) PREVENTIVE CARE PLATFORM FOR INTERACTIVE PATIENT MONITORING

(71) Applicant: ETS Platform, LLC, Coral Gables, FL (US)

(72) Inventors: David Hold, Ma'ale Shomron (IL); Tiberiu Weisz, Jamaica, NY (US)

(21) Appl. No.: 16/966,327

(22) PCT Filed: Jan. 30, 2019
(86) PCT No.: PCT/US2019/015747

§ 371 Date: Jul. 30, 2020

Related U.S. Application Data
(60) Provisional application No. 62/624,337, filed on Jan. 31, 2018.

(51) Int. Cl. G16H 80/00; G16H 10/60; G16H 20/10; G16H 40/67; A61B 5/00; A61B 5/145

(52) U.S. Cl. CPC G16H 80/00; G16H 10/60; G16H 20/10; G16H 40/67; A61B 5/4833; A61B 5/14532; A61B 5/4082; A61B 5/0006; A61B 5/746; A61B 5/4818

(57) ABSTRACT

An interactive platform providing health care prevention is provided. The platform includes a plurality of doctor interfaces, each doctor interface being associated with a doctor or other health care provider participating in the platform, patient interfaces coupled to peripherals measuring and determining patient parameters associated with the patients' health, said patient interfaces receiving, patient information, instructions, data from peripherals, and so on. In one embodiment, each patient interface is associated with a particular patient. In another embodiment, an intermediate assistant is provided with a patient interface adapted to provide information to and receive information related to several patients. A master server in communication with the doctor and patient interfaces and adapted to collect information and present information through said interfaces. Rules and criteria are used by the master server to monitor the patients' parameters, send reminders to the patient interfaces, and multi-level alarms to the doctor interfaces and devices of third parties when the patient parameters do not meet criteria selected by the individual doctors for their patients.

### BACKGROUND OF THE INVENTION

### A. Technical Field

This invention presents a comprehensive fully automated platform servicing from a master server a plurality of doctors (or health care provider) and patients, each patient being associated with one or more peripherals provided to monitor a critical patient parameter, preferably the peripherals or off the shelf devices. Each doctor is associated with at least one patient and each patient is associated with a respective patient interface. The doctor sets up including criteria such as ranges, thresholds and/or other limiting values or other criteria for measurements obtained from the peripherals, as well as certain operational rules. These rules and criteria are saved in a memory for a master server communicating with the patient interfaces, as well as interfaces associated with the doctors, via conventional secure communication channels. These rules and criteria define abnormal conditions for the patients and when such conditions are detected, the master server generates appropriate multi-level alerts to the respective doctor, patient care taker and/or other personnel. The platform provides many other functions designed to enable a doctor to provide preventive care to patients on a one to one basis or a remote mode in which a doctor or health clinic can monitor and provide preventive care to many patients.

### B. Description of the Prior Art

Individual devices are available from many vendors that can be used to monitor various patient parameters. The devices can then be coupled to communication channels to record information and/or send information to a remote location. However, there are no systems presently available that can provide a comprehensive interactive means of monitoring patient health and provide useful, on demand information about patient's medical condition using criteria selected by doctors or other health care providers.

### SUMMARY OF THE INVENTION

The present platform presents a comprehensive all-inclusive system that can be used by a plurality of doctors, hospitals, clinics and other health care providing entities to monitor a plurality of patients on individual basis or via a field located health care assistant to monitor and provide preventive care to several patients. The later mode is particularly effective for providing health care for indigent people or people located to remote areas with no immediate access to health care facilities.

The platform includes a plurality of doctor interfaces, each doctor interface being associated with a doctor and other health care provider participating in the platform, patient interfaces coupled to peripherals measuring and determining patient parameters associated with the patients' health, the patient interfaces receiving, patient information, instructions, data from peripherals, and so on, associated with a particular patient, or several patients. A master server in communication with the doctor and patient interfaces is adapted to collect information and present information through said interfaces. Rules and criteria are used by the master server to monitor the patients' parameters, send reminders to the patient interfaces, and send multi-level alarms to the doctor interfaces and interfaces of third parties when the patient parameters do not meet criteria selected by the individual doctors for their patients.

The platform is modular to provide a large number of patients and respective health care providers with customized monitoring peripherals and support for the same. Of course, not all the features described need to be provided for each patient and/or health care provider. Importantly, all data exchange is performed using protocols that meet all the HIPAA regulations and regulations of other entities concerned with patient data security and patient privacy.

### BRIEF DESCRIPTION OF THE DRAWINGS

FIG. 1 shows a block diagram illustrating the various elements of the platform constructed in accordance with this invention.

FIG. 2A shows the registration operation on the dashboard.

FIG. 2B shows the operation of the patient device.

FIG. 2C shows the running operation of the dashboard.

### DESCRIPTION OF THE INVENTION

In this application, the term PATIENT PARAMETER is used to designate any physical characteristic related directly or indirectly to the health or medical condition of a patient. Examples of such parameters are weight, temperature, blood pressure, blood glucose content, drug regimen, and so on.

The term PATIENT INTERFACE refers to a device associated with the patient (or field operator) for receiving parameters and transmitting them to the master server, and to receive information, instructions and other data for presentation to the patient. The patient interface may be a smart cell phone, a tablet, or other similar device.

The term DOCTOR INTERFACE refers to a device associated with a doctor, a nurse, or other health care provider for presenting alarms and other information related to the respective patients being treated, and for receiving and transmitting instructions, information, data, etc. to the patient interface and/or the master server. The doctor interface may be implemented using a desk top computer, a smart phone, a tablet, etc.

The term MASTER SERVER refers to a server that exchanges information from the patient interfaces and the doctor interfaces and generates respective commands, information, etc., to the patient and doctor interfaces via secure HIPAA compliant communication channels.

The term DASHBOARD refers to a graphic interface presented on the doctor interface and used to collect data from the doctors, to present information to the doctors and to activate patient specific alarms in response to the individual criteria set by each doctor for each patient for at least some of the patient parameters.

The term CRITERIA refers to a set of rules defining thresholds and other patient specific data used to generate alarms and affect the operation of the platform. An important aspect of the present invention is that each doctor selects his or set of rules for each of his/her patient.

The term PLATFORM refers to the combination of the patient, interfaces, patient interfaces, master server and software required to operate the same. The terms platform and system are used interchangeably.

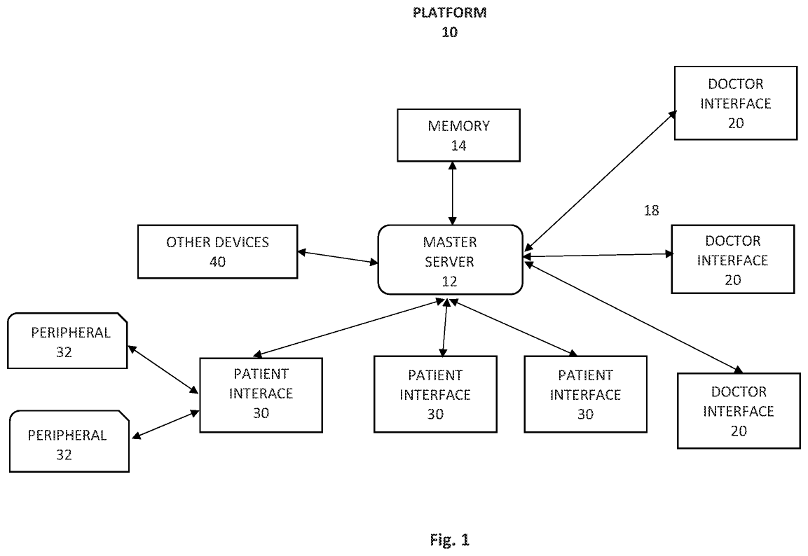

FIG. 1 shows the various components of platform 10. The platform includes a master server 12. The master server is associated with a memory bank 14 that stores the criteria and other data required for the operation of the platform 10, as well as the information obtained from the patients, the doctors, etc. The master server is adapted to generate a visual interface referred to as a dashboard 16 used as described below. The master server is connected by a plurality of standard communication channels 18 to a plurality of doctor interfaces 20, patient interfaces 30 and other devices 40.

The patient devices 30 are in communication via standard protocols (such as Bluetooth) to one or more peripherals 32, each peripheral being adapted to obtain a respective patient parameter associated with a respective patient, such as weight, heart rate, cardiac function, sleep-related parameters, blood pressure, blood glucose content, etc. As mentioned above, the patient interfaces 30 are implemented standard devices such a smartphones, tablets, etc., having means of presenting information to the patient, such as a screen, and speakers, and receiving information from the patient, such as keyboard or touchscreen. The patient devices further include peripheral interface components for communicating with the patient peripherals.

The doctor interfaces 20 are implemented standard devices such a smartphones, tablets, etc., having means of presenting information to the doctor, such as a screen, and speakers, and receiving information from the doctor, such as keyboard or touchscreen.

The devices 40 are used to communicate with other individuals or entities as discussed below and may include devices associated with emergency personal such as EMS.

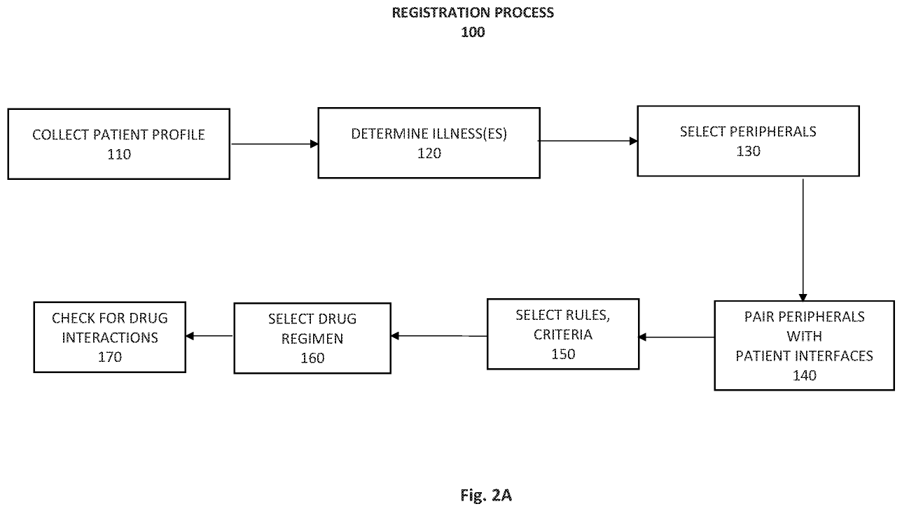

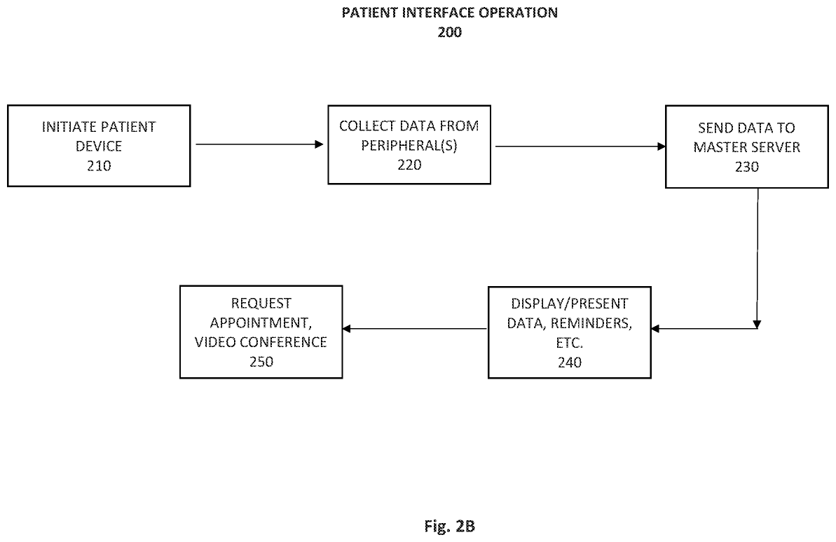

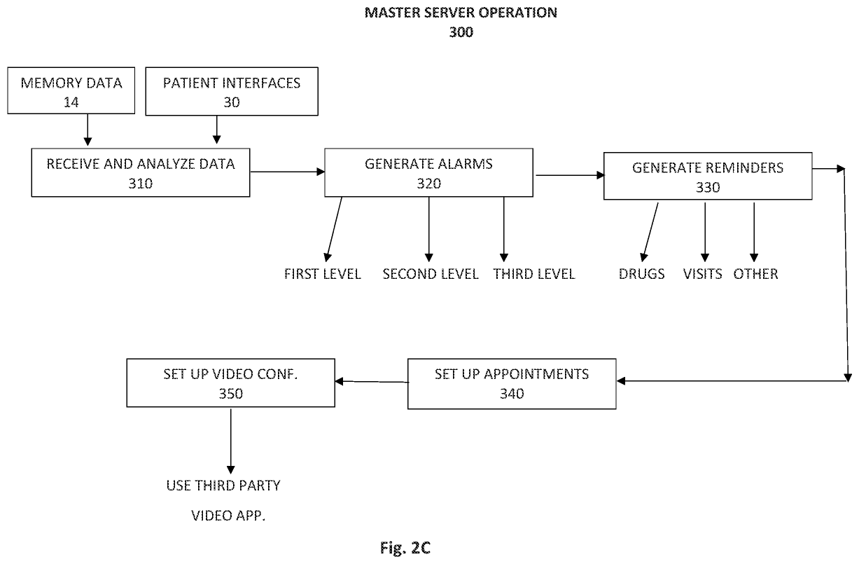

FIGS. 2A-2C show the operations and functionalities of the platform 10, as well as the operations of its components. More particularly, FIG. 2A, shows the registration process 100 used to register a particular patient on platform 10.

Each doctor and/or his nurse, medical assistant, etc. initiates the registration process 100 on a doctor device 20 during which each patient is registered and equipped. This process includes obtaining data from the patient that defines his profile, including ID, address, doctors, basic medical data such as current weight, heart rate, ECG, blood pressure, blood glucose level etc. (step 110). An initial diagnosis is also made to determine what is/are the patient's ailments (step 120). This information is used to select what patient parameters need to be monitored. For example, a patient may be diagnosed as a diabetic and the parameters that will be monitored will be his weight and glucose level. The appropriate monitoring devices are then assigned to the patient (step 130), set up and paired with the patient device (step 140). The patient can provide his own patient interface 30 or a patient interface may be provided by the doctor as needed. Preferably the patient interface is either preloaded with, or an appropriate app (e.g., based on 10S, Android, etc.) is downloaded into the patient interface.

The doctor also sets up the appropriate criteria specifically tailored to each patient based on the doctor's individual preferences and experience. For example, if the current weight of the patient is 80 Kg, the doctor may set a high weight threshold as 90 Kg and a low threshold of 75 Kg. Importantly, when the criteria are assigned by the doctor, the doctor may be provided with corresponding standard thresholds that are well known in the art to insure that he does not make a mistake. The doctor can then look at the standard thresholds and if they are too different from the thresholds he has selected, he may change them (step 150).

The doctor can also set up a regimen of drugs for the patient including one or more drugs, the amount, frequency etc. The current drugs taken by the patient is also recorded as part of the registration process (step 160). This information is compared against one or more appropriate interaction data bases to insure that there are no dangerous interactions between these drugs (step 170). If an interaction is detected, the doctor is notified immediately so that he can change the drug regimen to avoid such interaction. It should be noted that each time data (including criteria, etc.) is entered or changed for any patient, the data, time and author of such data/change of data is recorded for future reference. In this way, if a mistake is made, it can be traced.

The registration process 100 is implemented using the dashboard 16. For this purpose, the dashboard may include several pages that are presented on the respective device 20 providing information to the doctor and requesting the necessary inputs.

The patient interface operation 200 is described broadly in FIG. 2B. At the designated times, the patient initiates his device 210. The device then starts by presenting to the patient the various tests are available to him. For example, a diabetic may be asked for his weight and blood glucose level. The patient then activates the respective peripherals 32, and the respective data is collected (step 220), optionally presented to the patient, and is then transmitted to the master server 10 via the appropriate channel 18 (step 230). As indicated above, preferably each peripheral 32 is coupled to the respective patient interface 30 and each measurement taken by such peripheral is automatically transmitted to the patient interface 20. In one embodiment, the measurement is displayed to the patient and the patient then actively instructs the patient interface to forward the measurement to the master server 12. In another embodiment, the patient interface 20 receives the measurement and automatically transmits it to the master server 12. However, the measurement is still optionally displayed to the patient on his interface 30. In some instances, it may be preferable for the patient to enter the respective measurement manually. Optionally, several measurements can be displayed on the patient interface 20 either automatically or on demand, in historical order (step 240).

Importantly, the patient interface 30 is also used for other functions, including receiving reminders or other information from the doctors, requesting appointments with the doctors, requesting video conferencing etc., as discussed below (step 250).

The general operation of the master server 12 is described in FIG. 2C. The master server 12, during normal operation, first collects data from the patient interfaces 30 and stores them in memory 14. The server 12 also analyzes the collected data and determines if there is any action is required, based on the criteria selected by the respective doctor (step 310). For example, if a received patient parameter, e.g. weight of a patient is within prescribed limits, it is merely recorded. However, if the received parameter is outside the prescribed limits, an alarm is generated. Preferably the alarms are classified into several levels, depending on several factors such as the deviation of the received parameter from the prescribed ranges, response or lack of response from a patient or a doctor, the temporal frequency that the respective patient parameter was outside the desired range and so on (step 320). In one embodiment, a first or low level is sent to the respective doctor device 20 (and optionally to other personnel designated by the doctor or the patient, via a device 40). If the deviation is large, or if the doctor does not respond within a preset period, a second alarm is generated and sent out. If there is still no response from the doctor acknowledging the alarm and if the situation requires it, a third alarm may be sent as required.

The alarms may be sent to the same or to different personnel, including medical emergency services. The alarm is sent in the form of SSM message, an email or other electronic means and can include information identifying the patient, the received parameter, the acceptable parameter ranges, who has been alerted and so on.

Drug reminders are handled in a similar fashion. At predetermined times related to the respective drug regimen, the patient receives a reminder to take his drug (steps 330, 240). He is then expected to respond with either an acknowledgement or an indication of whether he will take the drug or not. If an acknowledgment or response is not received in timely fashion, a second reminder and then a third reminder may be sent (step 330). If there is still no acceptable response, an alarm may be sent to the physician device 20, and other personnel and caretakers (via devices 40) indicating noncompliance (step 320). The process 300 is extended to critical patient parameters and incidents as well. For example, if the heart rate is too high or too low, the glucose level is too high, other cardiac functions are abnormal, and so on, alarms are sent not only to the doctor but to third parties such as secondary care providers, children of the patient, EMS services etc.

Platform 10 may be used by the patient to request an appointment with the doctor and an appointment is then set based on the doctor's calendar and availability (step 340). The patient may optionally request a video conference with the doctor. The doctor may then schedule the conference with the patient and at the appropriate time initiate the conference using, for example a third-party video conferencing application (step 350).

In one embodiment, the patient interface is modified to provide transportation to the patient, using a service such as a taxi or similar system. For this purpose, a virtual button on device 20 is provided for calling a car. When this routine is initiated, a standard call goes out to an associated car service indicating the address of the patient, and the destination. Optionally, the patient can request a return service a predetermined time after pick up. The car service then sends a car for the patient.

Preferably sleep apnea may be treated using two separate peripherals 32 that require two different accessories. The first peripheral 32 is the sleep apnea identification component and is used to determine whether the patient has this condition. The second component is the compliance component is used to monitor when the sleep apnea device is used at night. This information is reported and used to determine whether the patient is compliant or not as follows:

1. The doctor prepares a CPAP device for the patient and provides instructions for the use of the same. As part of this preparation, the doctor provides the usage rules for the device, such as use every night for at least six hours. This information is entered on the profile of the patient, using the dashboard. The doctor also sets alert levels for the use of sleep apnea peripheral.

2. The patient takes home the CPAP device, and any other accessories required by the doctor, such as a cardiac monitor, oximeter, etc.

3. The patient uses the CPAP device and any other accessories as required.

4. The CPAP device generates an alert every time the patient starts and stops using the CPAP device (or generates a report at the end of each session with the CPAP device indicating when the CPAP device was activated and deactivated).

5. The alerts/report is sent to the server 12 and recorded.

6. If the CPAP device usage does not conform to the regime set up by the doctor, an alert is sent to the doctor and/or others using protocol described above. The reliability of the compliance component can be substantially increased if the CPAP device includes some means of identifying be separate and independent measurements the person using for example a biometric sensor.

In another embodiment, educational information is provided by server 12 to the patient via patient interface 30. For example, if the doctor thinks it's appropriate, the patient is sent a reminder at regular intervals for a breast self examination, with instructions for performing such an examination.

In one embodiment, the patient is a pregnant mother provided with a patient interface 30 and a pregnancy monitoring peripheral 32 that measures fetal heart rate, fetal movement, contraction of the uterus and the mother's heart rate.

In some instances, for example in rural areas, it may be inappropriate to provide each patient with his or her patient interface 30 and peripherals. In such cases, field operators are used who then visits each patient at regular intervals or on demand. Each field operator is equipped with a respective patient device and peripherals.

The platform performs one or more of the following functions:

a. Registering physician and their patients, which then establishes both a physician file and patient electronic record file:

b. Overseeing the monitoring of the patient by receiving, analyzing, storing and relaying pertinent information to the respective doctors, and dynamically and seamlessly changing thresholds or other operational parameters of a patient as necessary from the doctor interfaces without requiring another visit to the physician by the patient. The peripherals are used to monitor one or more of the following patient parameters: Diabetic Glucose Monitoring; Blood Pressure Monitoring; Pulse Oxygen Saturation; Medication Management; Weight Monitoring; PTI/NR Testing; Cardiac performance; Sleep Apnea; Pain management; Mood monitoring; Physical conditions related to tremors indicative of Parkinson and other diseases; Psychological monitoring; Pregnancy monitoring.

c. Managing monitoring peripheral and other hardware and equipment inventories.

d. Developing rating for the participating physicians including their response times.

e. Minimizing hospital readmission event and enabling faster patient interaction and diagnosis for hospitals and emergency staff by providing access to the patient records through his interface.

f. Monitoring drug regimen compliance.

g. Preventing undesirable drug interactions.

h. Enabling field heath care providers to treat patients remote from doctors.

i. Monitoring the use of sleep apnea preventive devices such as CPAP devices.

j. Providing video conferencing between one or more doctors and the patients

k. Generating alarms at different levels commensurate with current patient information

l. Pregnancy monitoring

m. Providing information to a patient for self care, such as self breast examination.

Numerous modifications may be made to the invention without departing from its scope as defined in the appended claims.

We claim:

1. An interactive doctor patient interface platform enabling doctors to monitor and communicate with a plurality of patients having specific characteristics diseases, each patient being associated with one or peripherals measuring the parameters associated with a disease of that patient and each patient being under the supervision of a health care provider for a particular diseases, said platform comprising:
a plurality of doctor interfaces adapted to present and receive information and commands, each doctor interface being associated with a doctor, each doctor supervising at least one patient;
a plurality of patient interfaces adapted to present and receive information, each patient interface communicating with one of said patient peripherals and being associated with a respective patient; and
a master server communicating with all the peripherals of all the patients via said patient interfaces to receive information indicative of said parameters, said master server being configured to analyze said information, determine if said parameters meet a predetermined criteria set by the respective doctor of the respective patient, and selectively generate at least a first and a second alert sequentially based on said analysis, the doctor interfaces selectively presenting information about their respective patients on demand, said master sever being adapted to generate the second alert if said first alert is not acknowledged.

2. The platform of claim 1 wherein said patient interfaces are one of a tablet and a smartphone.

3. The platform of claim 1 wherein said master server is configured to generate a regimen for each patient for obtaining said parameters or take medicine, said master server being further adapted to contact said patient interfaces to determine if said patients have complied with said regimen, and to generate alerts to the physicians if there is lack of compliance by some patients.

4. The platform of claim 3 wherein said master server is configured to send queries to said patient interfaces to determine compliance.

5. The platform of claim 1 wherein said doctor interfaces are adapted to provide patient parameters obtained from a plurality of said patients in sequence when requested by a doctor, said patient parameters being presented in chronological order.

6. The platform of claim 5 wherein said parameters include electrocardiograms (EKGs).

7. The platform of claim 6 further comprising a memory containing information about interactions between different drugs prescribed to patients, wherein said platform is configured to receive drug prescriptions for a patient and to check with said memory to determine for each new drug whether said new drug will interact with another drug prescribed for that patient.

8. The platform of claim 7 wherein said physician requests a change in a drug selected from one of a drug kind and drug dosage, wherein said platform is adapted to check said database to determine any conflicts with the new drug and any other existing drugs and/or conditions of the patient.

9. The platform of claim 1 further comprising a test feature prompting a patient to perform a test and once test data is provided to the master server, if the patient is not performing the test than then a notification feature is activated to alert a designated person that the patient has failed to follow the test.

10. The platform of claim 1 wherein said patient interfaces are adapted to receive manual inputs from patients when automatic transmitting of information to the platform is unavailable.

11. The platform of claim 10 wherein said master server keeps track of manual entries from patients, and to generate an alert when manual entries from a patient exceed a predetermined limit.

12. The platform of claim 1 wherein at least one peripheral is adapted to monitor sleep apnea related parameters in a patient and to generate corrective steps to stop sleep apnea in the patient.

13. The platform of claim 1 wherein at least one a peripheral and a patient interface include an accelerometer, said peripheral or patient interface being adapted to sense indications in a Parkinson's disease in a patient when the patient is holding the same.

14. The platform of claim 1 further including an alarm button disposed on one of said patient interfaces and selectively activated by the patient, wherein in response to the activation of the alarm button, the master server initiates an alarm protocol including one of sending a message to a caregiver, Emergency Medical Services (EMS) and a health care provider.

15. The platform of claim 14 wherein the alarm button is further adapted to be activated automatically in response to a predetermined condition, such as the patient falling down.

16. The platform of claim 14 wherein the alarm button further includes geographic location elements for selectively locating the patient and transmitting said location to a remote party.
